SEQUENCE LISTING (13 sequences)

SEQ ID NO: 1 - protein, 483 aa, Homo sapiens
MSGHKCSYPWDLQDRYAQDKSVVNKMQQKYWETKQAFIKATGKKEDEHVVASDADLDAKLELFHSIQRTCLDLSKAIVLYQKRICFLSQEENELGKFLRSQGFQDKTRAGKMMQATGKALCFSSQQRLALRNPLCRFHQEVETFRHRAISDTWLTVNRMEQCRTEYRGALLWMKDVSQELDPDLYKQMEKFRKVQTQVRLAKKNFDKLKMDVCQKVDLLGASRCNLLSHMLATYQTTLLHFWEKTSHTMAAIHESFKGYQPYEFTTLKSLQDPMKKLVEKEEKKKINQQESTDAAVQEPSQLISLEEENQRKESSSFKTEDGKSILSALDKGSTHTACSGPIDELLDMKSEEGACLGPVAGTPEPEGADKDDLLLLSEIFNASSLEEGEFSKEWAAVFGDGQVKEPVPTMALGEPDPKAQTGSGFLPSQLLDQNMKDLQASLQEPAKAASDLTAWFSLFADLDPLSNPDAVGKTDKEHELLNA

SEQ ID NO: 2 - protein, 18 aa, synthetic construct
EGQQIGILISLEEENQRK

SEQ ID NO: 3 - protein, 29 aa, synthetic construct
YLQNICLEMKANSDIKMLQKGKEGQQIGI

SEQ ID NO: 4 - protein, 512 aa, synthetic construct
MSGHKCSYPWDLQDRYAQDKSVVNKMQQKYWETKQAFIKATGKKEDEHVVASDADLDAKLELFHSIQRTCLDLSKAIVLYQKRICFLSQEENELGKFLRSQGFQDKTRAGKMMQATGKALCFSSQQRLALRNPLCRFHQEVETFRHRAISDTWLTVNRMEQCRTEYRGALLWMKDVSQELDPDLYKQMEKFRKVQTQVRLAKKNFDKLKMDVCQKVDLLGASRCNLLSHMLATYQTTLLHFWEKTSHTMAAIHESFKGYQPYEFTTLKSLQDPMKKLVEKEEKKKINQQESTDAAVQEPSQYLQNICLEMKANSDIKMLQKGKEGQQIGILISLEEENQRKESSSFKTEDGKSILSALDKGSTHTACSGPIDELLDMKSEEGACLGPVAGTPEPEGADKDDLLLLSEIFNASSLEEGEFSKEWAAVFGDGQVKEPVPTMALGEPDPKAQTGSGFLPSQLLDQNMKDLQASLQEPAKAASDLTAWFSLFADLDPLSNPDAVGKTDKEHELLNA

SEQ ID NO: 5 - DNA, 54 bp, synthetic construct
  source 1..54: mol_type = other DNA
gaaggccagcaaattggcatattaatttcattagaggaagaaaaccagcgcaag

SEQ ID NO: 6 - DNA, 87 bp, synthetic construct
  source 1..87: mol_type = other DNA
tatttacagaatatttgcctcgagatgaaagcaaattcagatattaagatgctacagaaagggaaagaaggccagcaaattggcata

SEQ ID NO: 7 - DNA, 1539 bp, synthetic construct
  source 1..1539: mol_type = other DNA
atgtcaggacacaaatgcagttatccctgggacttacaggatcgatatgctcaagataagtcagttgtaaataagatgcaacagaaatattgggagacgaagcaggcctttattaaagccacagggaagaaggaagatgaacatgttgttgcctctgacgcggacctggatgccaagctagagctgtttcattcaattcagagaacctgtctggacttatcgaaagcaattgtactctatcaaaagaggatatgtttcttgtctcaagaagaaaacgaactgggaaaatttcttcgatcccaaggtttccaagataaaaccagagcaggaaagatgatgcaagcgacaggaaaggccctctgcttttcttcccagcaaaggttggccttacgaaatcctttgtgtcgatttcaccaagaagtggagacttttcggcatcgggccatctcagatacttggctgacggtgaaccgcatggaacagtgcaggacggaatatagaggagcactattatggatgaaggacgtgtctcaggagcttgatccagacctctacaagcaaatggagaagttcaggaaggtacaaacacaagtgcgccttgcaaaaaaaaactttgacaaattgaagatggatgtttgtcaaaaagtggatcttcttggagcgagcagatgcaatctcttgtctcacatgctagcaacataccagaccactctgcttcatttttgggagaaaacttctcacactatggcagccatccatgagagtttcaaaggttatcaaccatatgaatttactactttaaagagcttacaagaccctatgaaaaaattagttgagaaagaagagaagaagaaaatcaaccagcaggaaagtacagatgcagccgtgcaggagccgagccaatatttacagaatatttgcctcgagatgaaagcaaattcagatattaagatgctacagaaagggaaagaaggccagcaaattggcatattaatttcattagaggaagaaaaccagcgcaaggaatcctctagttttaagactgaagatggaaaaagtattttatctgccttagacaaaggctctacacatactgcatgctcaggacccatagatgaactattagacatgaaatctgaggaaggtgcttgcctgggaccagtggcagggaccccggaacctgaaggtgctgacaaagatgacctgctgctgttgagtgagatcttcaatgcttcctccttggaagagggcgagttcagcaaagagtgggccgctgtgtttggagacggccaagtgaaggagccagtgcccactatggccctgggagagccagaccccaaggcccagacaggctcaggtttccttccttcgcagcttttagaccaaaatatgaaagacttacaggcctcgctacaagaacctgctaaggctgcctcagacctgactgcctggttcagcctcttcgctgacctcgacccactctcaaatcctgatgctgttgggaaaaccgataaagaacacgaattgctcaatgcatga

SEQ ID NO: 8 - RNA, 12 nt, Homo sapiens
  source 1..12: mol_type = other RNA
tgtgtgtgtgtg

SEQ ID NO: 9 - protein, 11 aa, synthetic construct
LISLEEENQRK

SEQ ID NO: 10 - DNA, 19 bp, synthetic construct
  source 1..19: mol_type = other DNA
ttccgttttgaacatgcaa

SEQ ID NO: 11 - DNA, 25 bp, synthetic construct
  source 1..25: mol_type = other DNA
cacacacacacacacacacacacac

SEQ ID NO: 12 - DNA, 19 bp, synthetic construct
  source 1..19: mol_type = other DNA
  modified_base 1..2: mod_base = OTHER; note = beta-D-oxy-locked nucleic acid
  modified_base 1..19: mod_base = OTHER; note = phosphorothioate internucleoside linkages
  modified_base 16..19: mod_base = OTHER; note = beta-D-oxy-locked nucleic acid
  modified_base 4: mod_base = m5c
  modified_base 17: mod_base = m5c
ttccgttttgaacatgcaa

SEQ ID NO: 13 - RNA, 25 nt, synthetic construct
  source 1..25: mol_type = other RNA
  modified_base 1..25: mod_base = OTHER; note = 2'-O-methoxyethyl nucleoside
  modified_base 1..25: mod_base = OTHER; note = phosphorothioate internucleoside linkages
  modified_base 1: mod_base = m5c
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
  modified_base 19: mod_base = m5c
  modified_base 21: mod_base = m5c
  modified_base 23: mod_base = m5c
  modified_base 25: mod_base = m5c
cacacacacacacacacacacacac

(19) United States
(12) Patent Application Publication
(10) Pub. No.: US 2026/0210980 A1
(43) Pub. Date: Jul. 23, 2026

(54) PEPTIDE BIOMARKER

(71) Applicant: Hoffmann-La Roche AG, Little Falls, NJ (US)

(72) Inventors: Marco TERRIGNO, Memphis, TN (US); Jonas VIKESAA, Fredensborg (DK); Congwei WANG, New Haven, CT (US)

(21) Appl. No.: 19/629,210

(22) Filed: Mar. 26, 2026

Related U.S. Application Data
(63) Continuation of application No. PCT/EP2024/077048, filed on Sep. 26, 2024.

(30) Foreign Application Priority Data
Sep. 29, 2023 (EP) 23200991.0

(51) Int. Cl. G01N 33/68; C07K 16/18

(52) U.S. Cl. CPC G01N 33/6896; C07K 16/18; G01N 2570/00; G01N 2800/28; G01N 2800/2814; G01N 2800/50; G01N 2800/52

(57) ABSTRACT

The present invention relates to a splice variant of an ICA1 protein that acts as a biomarker for a TDP-43 pathology. In particular, the present invention relates to methods for identifying a splice variant of ICA1 comprising a cryptic peptide sequence, and to related methods of identifying a TDP-43 pathology and/or reduced TDP-43 function in a subject.

### SEQUENCE LISTING

The instant application contains a Sequence Listing which has been submitted electronically in XML format and is hereby incorporated by reference in its entirety. Said XML copy, created on Mar. 19, 2026, is named 51551-030001_Sequence_Listing_3_19_26 and is 23,063 bytes in size.

### FIELD OF THE INVENTION

The present invention relates to a splice variant of an ICA1 protein that acts as a biomarker for a TDP-43 pathology. In particular, the present invention relates to methods for identifying a splice variant of ICA1 comprising a cryptic peptide sequence, and to related methods of identifying a TDP-43 pathology and/or reduced TDP-43 function in a subject.

### BACKGROUND TO THE INVENTION

TAR DNA binding protein 43 (TDP-43) is a versatile RNA/DNA binding protein involved in RNA-related metabolism. Dysregulation of TDP-43 deposits act as inclusion bodies in the brain and spinal cord of patients with the motor neuron diseases: amyotrophic lateral sclerosis (ALS) and frontotemporal lobar degeneration (FTLD) (Prasad et al., Front. Mol. Neurosci., 2019).

TDP-43 is predominantly localized in the nucleus but also shuttles to the cytoplasm for some of its functions (Ayala et al., 2008). In several neurodegenerative diseases TDP-43 is a common denominator. In disease, such as in ALS and FTLD, there is an increase in the cytoplasmic TDP-43 concentration leading to cytoplasmic inclusion formation (Neumann et al., 2006; Winton et al., 2008a). The cytoplasmic mislocalization can be associated with nuclear depletion, resulting in a reduction or loss of TDP-43 function. For example, more than 95% of ALS patients display pathological mislocalization of TDP-43 and several mutations in its gene cause familial ALS. There are TDP-43 mutations which result in aberrant splicing of TDP-43 target RNAs, resulting in widespread splicing aberration (see for example Arnold et al., PNAS 2013 110 E736-745 and Yang et al., PNAS. U.S.A. 111, E1121-E1129).

Klim et al., reports that STMN2 loss upon reduced TDP-43 function is due to altered STMN2 splicing, and suggests restoring STMN2 as a therapeutic strategy for ALS.

TDP-43 depletion is indicated in a range of diseases, referred to as TDP-43 pathologies, and including for example diseases such as amyotrophic lateral sclerosis (ALS), frontotemporal lobar degeneration (FTLD), Progressive supranuclear palsy (PSP), Primary lateral sclerosis, Progressive muscular atrophy, Alzheimer's disease, Parkinson's disease, autism, Hippocampal sclerosis dementia, Down syndrome, Huntington's disease, polyglutamine diseases, such as spinocerebellar ataxia 3, myopathies and Chronic Traumatic Encephalopathy.

Tollervey et al., Nature Neuroscience 2010, 452-458 reports on the characterization of the RNA targets and position dependent splicing regulation of TDP-43 in healthy brain tissue and brain tissue from FTLD patients. Most TDP-43 binding sites mapped to introns, long non-coding RNAs (lncRNA) and intergenic transcripts, which were enriched for UG-rich motifs. The conserved RNP segments in TDP-43 are involved in binding to TAR DNA sequences and RNA sequences with UG-repeats (Ayala et al., J. Mol. Biol. 2005; 348:575-588). TDP-43 depletion in cells, such as in TDP-pathologies is correlated to the loss of RNA binding of TDP-43 to TDP-43 RNA targets.

TDP-43 binding sites in human RNAs are available online from the A database of RNA binding proteins and associated moTifs-see https://attract.cnic.es/results/e9f29380-8921-406e-84a8-27ce9b9398b4 #. Certain characterized human RNA TDP-43 binding sites disclosed include the following RNA sequences: GUGAAUGA, GUUGUGC, UGUGUGUGUGUG, GAAUGG, UGUGUGUG, GAAUGA, UGUGUG, GUUGUUC, and GUUUUGC.

The role of TDP-43 as a causative agent of a pathology can typically only be identified post-mortem.

Thus, there is a need for biomarkers which can be used to diagnose patients as having a TDP-43 pathology. This would enable patients to be diagnosed and treatment prior to disease progression.

### SUMMARY OF THE INVENTION

The present inventors have surprisingly found that a splice variant of ICA1 comprising a cryptic peptide sequence acts as a biomarker for loss of TDP43 expression and/or function, and thus can be used as a diagnostic tool for identifying diseases/pathologies associated with TDP43.

The present invention provides a method for identifying a TDP-43 pathology and/or reduced TDP-43 function in a subject, comprising the step of identifying a splice variant of ICA1 in a sample obtained from the subject.

The present invention provides a method of identifying a subject that is at risk of developing a TDP-43 pathology and/or reduced TDP-43 function, wherein the method comprises a step of identifying a splice variant of ICA1 in a sample obtained from the subject, wherein the presence of the splice variant of ICA1 in the sample indicates that the subject is likely to develop a TDP-43 pathology.

The present invention provides a method for determining whether a subject suffering from a TDP-43 pathology and/or reduced TDP-43 function is likely to respond to a treatment, wherein the method comprises a step of identifying a splice variant of ICA1 in a sample obtained from the subject, wherein the presence of the splice variant of ICA1 in the sample indicates that the subject is likely to respond to a treatment.

The present invention provides a method for monitoring the therapeutic success of a treatment of a TDP-43 pathology and/or reduced TDP-43 function in a subject, wherein the method comprises a step of identifying a splice variant of ICA1 in a sample obtained from the subject; and further comprises a step of predicting the therapeutic success of the treatment of the TDP-43 pathology and/or reduced TDP-43 function based on the detection of the splice variant of ICA1.

In some embodiments, the treatment may comprise an antisense oligonucleotide capable of restoring the functional phenotype of one or more TDP-43 target RNA(s) in a cell which is TDP-43 depleted, or is expressing aberrant TDP-43 protein.

The present invention provides a use of a splice variant of ICA1 as a biomarker for a TDP-43 pathology and/or reduced TDP-43 function.

In some embodiments, the splice variant of ICA1 may comprise a cryptic peptide sequence.

In some embodiments, the splice variant of ICA1 may comprise a cryptic peptide sequence of 10-50 amino acids.

In some embodiments, the splice variant of ICA1 may comprise a cryptic peptide sequence of 25-35 amino acids.

In some embodiments, the splice variant of ICA1 may comprise a cryptic peptide sequence of 29 amino acids.

In some embodiments, the cryptic peptide sequence may be inserted at a position corresponding to position 302 of the amino acid sequence according to SEQ ID NO: 1 encoding wild-type ICA1.

In some embodiments, the cryptic peptide sequence may comprise or consist of the amino acid sequence YLQNICLEMKANSDIKMLQKGKEGQQIGI (SEQ ID NO: 3), or a variant thereof having at least 80% sequence identity thereto.

In some embodiments, the cryptic peptide sequence may comprise or consist of an amino acid sequence having at least 85%, 90%, 95%, 96%, 97%, 98%, 99%, or 100% sequence identity to the amino acid sequence YLQNICLEMKANSDIKMLQKGKEGQQIGI (SEQ ID NO: 3).

In some embodiments, the cryptic peptide sequence may comprise or consist of the amino acid sequence YLQNICLEMKANSDIKMLQKGKEGQQIGI (SEQ ID NO: 3).

In some embodiments, the splice variant of ICA1 may comprise or consist of an amino acid sequence according to SEQ ID NO: 4 or a variant with at least 80% sequence identity thereto.

In some embodiments, the splice variant of ICA1 may comprise or consist of an amino acid having at least 85%, 90%, 95%, 96%, 97%, 98%, 99%, or 100% sequence identity to SEQ ID NO: 4.

In some embodiments, the splice variant of ICA1 may comprise or consist of the amino acid sequence according to SEQ ID NO: 4.

In some embodiments, the method may be an ex vivo method.

In some embodiments, the step of identifying the splice variant of ICA1 may comprise proteomic analysis or an antibody based assay.

In some embodiments, the step of identifying the splice variant of ICA1 may comprise measuring peptides in the sample using liquid chromatography (LC) and/or mass spectrometry (MS).

In some embodiments, the sample may be obtained from a bodily fluid or tissue, selected from: blood, serum, plasma, urine, saliva, brain tissue and cerebrospinal fluid (CSF).

In some embodiments, the subject may be human. In some embodiments, the subject may be a human patient.

In some embodiments, the TDP-43 pathology may be a neurological disorder.

In some embodiments, the TDP-43 pathology may be a neurological disorder selected from: amyotrophic lateral sclerosis (ALS), frontotemporal dementia, frontotemporal lobar degeneration (FTLD), progressive supranuclear palsy (PSP), primary lateral sclerosis, progressive muscular atrophy, alzheimer's disease, parkinson's disease, autism, hippocampal sclerosis dementia, down syndrome, huntington's disease, polyglutamine diseases, such as spinocerebellar ataxia 3, myopathies and chronic traumatic encephalopathy.

In some embodiments, the TDP-43 pathology may be a neurological disorder selected from: amyotrophic lateral sclerosis (ALS), frontotemporal dementia, and frontotemporal lobar degeneration (FTLD).

The present invention provides an antibody that binds to a splice variant of ICA1.

In some embodiments, the splice variant of ICA1 may comprise an epitope comprising or consisting of the amino acid sequence YLQNICLEMKANSDIKMLQKGKEGQQIGI (SEQ ID NO: 3), or a variant thereof having at least 80% sequence identity to thereto.

In some embodiments, the epitope may comprise or consist of an amino acid sequence having at least 85%, 90%, 95%, 96%, 97%, 98%, 99%, or 100% sequence identity to the amino acid sequence YLQNICLEMKANSDIKMLQKGKEGQQIGI (SEQ ID NO: 3).

In some embodiments, the splice variant of ICA1 may comprise or consist of an amino acid sequence according to SEQ ID NO: 4 or a variant with at least 80% sequence identity to SEQ ID NO: 4.

In some embodiments, the splice variant of ICA1 may comprise or consist of an amino acid having at least 85%, 90%, 95%, 96%, 97%, 98%, 99%, or 100% sequence identity to SEQ ID NO: 4.

The present invention provides a kit comprising at least one reagent for detecting a splice variant of ICA1, wherein the at least one reagent is an antibody according to the invention.

### BRIEF DESCRIPTION OF THE FIGURES

FIG. 1: Barplots showing the intensities of TDP-43 protein (A) or ICA1 peptide (EGQQIGILISLEEENQRK) (B) across different samples. n=3. Data are shown as the mean±standard deviation (SD).

FIG. 2: Structure of the LNA gapmer used for knocking down TDP-43.

FIG. 3: Structure of the (CA) n antisense oligonucleotide (ASO) (used for mimicking the function of TDP-43 and rescuing misspliced transcripts upon loss of TDP-43).

FIG. 4: Coding sequence of the splice variant of ICA1, showing the insertion of the cryptic exon at position 302.

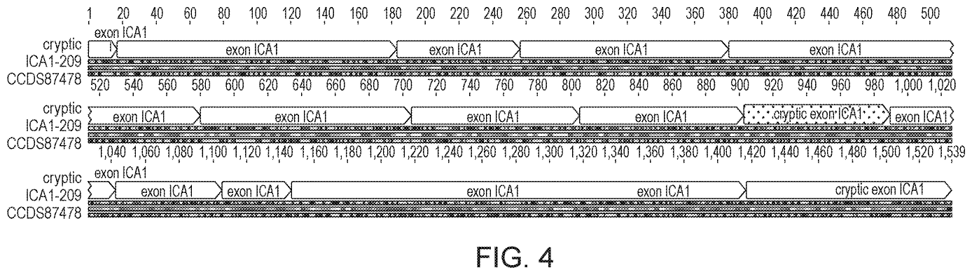

### DETAILED DESCRIPTION OF THE INVENTION

### TDP-43

TDP-43 is the TAR RNA/DNA binding protein which in humans is encoded on the human chromosome 1:11,012,653-11,022,858 forward strand (Gene ENSG00000120948, Chr 1:11, 012,344-11,025,739, example of a typical TDP-43 transcript=ENST00000439080.6), and is widely involved in RNA splicing, stability and metabolism.

In healthy cells the TDP-43 protein is located in the nucleus, however in several neurodegenerative diseases, dysfunction TDP-43 aggregates form in the cytoplasm (often associated with hyper-phosphorylated and ubiquitinated TDP-43).

TDP-43 is an example of a RNA binding protein which binds to GU repeats in numerous independent RNA transcripts. The interaction of RNA binding proteins, such as TDP-43 with the population of numerous RNA transcripts has a profound effect on the biology of the RNA transcripts, such as the splicing on pre-mRNA, RNA stability, RNA accumulation, and therefore provides a mechanism for effecting the expression of populations of independent RNAs in a cell.

This is of particular relevance in the case of TDP-43 depletion, where the loss of RNA binding of functional TDP-43 is closely associated with neurodegeneration, where the depletion of TDP-43 in neuronal cells results in a profound alteration in the RNA processing of a large population of RNA transcripts in the cell.

A TDP-43 pathology is a disease which is associated with reduced or aberrant expression of TDP-43 and/or reduced TDP-43 function, often associated by an increase in cytoplasmic TDP-43, particularly hyper-phosphorylated and ubiquitinated TDP-43.

TDP-43 depletion is indicated in a range of diseases, referred to as TDP-43 pathologies, and include for example amyotrophic lateral sclerosis (ALS), frontotemporal lobar degeneration (FTLD), Progressive supranuclear palsy (PSP), Primary lateral sclerosis, Progressive muscular atrophy Alzheimer's disease, Parkinson's disease, Autism, Hippocampal sclerosis dementia, Down syndrome, Huntington's disease, polyglutamine diseases, such as spinocerebellar ataxia 3, myopathies and Chronic Traumatic Encephalopathy.

Cells which are depleted in TDP-43 refer to cells where the functional level of TDP-43 is reduced. It will be understood that in TDP-43 pathologies, aberrant TDP-43 expression may result in accumulation of dysfunctional cytoplasmic TDP-43, and a reduction in the functional nuclear TDP-43 level—as such cells which are depleted in TDP-43 may be characterized by a reduction in the functional level of TDP-43, and may therefore be associated with an increase in the level of dysfunctional TDP-43.

For in vitro assessment, TDP-43 depletion may be engineered for example by genetic engineering approaches (e.g. CRISPR/CAS9), or as illustrated in the examples, by use of an antisense oligonucleotide inhibitor of TDP-43 (illustrated by a gapmer oligonucleotide targeting the human TDP-43 transcript).

In some embodiments, the cell which is depleted in TDP-43 is a neuronal cell.

### Splice Variant of ICA1

ICA1, also known as ICA69 or islet cell autoantigen 1, encodes a protein with an arfaptin homology domain found in the cytosol as well as on the Golgi complex and immature secretory granules. This protein may be an autoantigen in insulin-dependent diabetes mellitus and primary Sjogren's syndrome.

An illustrative ICA1 sequence is the human ICA1 sequence according to UniProt: Q05084-1. The amino acid sequence of human ICA1 (UniProt: Q05084-1) is provided below as SEQ ID NO: 1. As used herein, the amino acid sequence of human ICA1 (SEQ ID NO: 1) may be considered as a wild-type sequence, i.e. a canonical sequence.

-human ICA1
SEQ ID NO: 1
MSGHKCSYPWDLQDRYAQDKSVVNKMQQKYWETKQAFIKATGKKEDEHVVASDADLDAKLELFHSIQRTCLDLSKAI
VLYQKRICFLSQEENELGKFLRSQGFQDKTRAGKMMQATGKALCFSSQQRLALRNPLCRFHQEVETFRHRAISDTWL
TVNRMEQCRTEYRGALLWMKDVSQELDPDLYKQMEKFRKVQTQVRLAKKNFDKLKMDVCQKVDLLGASRCNLLSHML
ATYQTTLLHFWEKTSHTMAAIHESFKGYQPYEFTTLKSLQDPMKKLVEKEEKKKINQQESTDAAVQEPSQLISLEEE
NQRKESSSFKTEDGKSILSALDKGSTHTACSGPIDELLDMKSEEGACLGPVAGTPEPEGADKDDLLLLSEIFNASSL
EEGEFSKEWAAVFGDGQVKEPVPTMALGEPDPKAQTGSGFLPSQLLDQNMKDLQASLQEPAKAASDLTAWFSLFADL
DPLSNPDAVGKTDKEHELLNA

The present invention is based upon the surprising finding that a splice variant of ICA1 acts as a biomarker for loss of TDP-43 expression and/or function, and thus can be used as a diagnostic tool for identifying diseases/pathologies associated with aberrant TDP-43 expression and/or reduced TDP-43 function.

It will be understood that the term “a splice variant of ICA1” refers to an ICA1 protein that differs in its amino acid sequence to the amino acid sequence of a wild-type/canonical ICA1 protein, such as the human ICA1 protein having the amino acid sequence according to the sequence of SEQ ID NO: 1.

As such, the distinguishing feature between a wild-type ICA1 protein and a splice variant of ICA1 may be that the splice variant of ICA1 comprises an additional amino sequence, such as a cryptic peptide sequence, that is not present in the wild-type ICA1 protein. In other words, a splice variant of ICA1 may occur when a cryptic peptide sequence is inserted into the amino acid sequence of a wild-type ICA1 protein. The events that result in the formation of a splice variant may be known as mis-splicing or cryptic splicing.

In some embodiments, the splice variant of ICA1 may comprise a cryptic peptide sequence. In other words, the amino acid sequence encoding the splice variant of ICA1 may comprise the amino acid sequence encoding the wild-type ICA1 protein and may additionally comprise a further amino acid sequence which is a cryptic peptide sequence, that is not present in the amino acid sequence encoding the wild-type ICA1 protein.

As used herein, the term “cryptic peptide sequence” can be considered as an amino acid sequence that is introduced (e.g. inserted) into the amino acid sequence encoding the wild-type ICA1 protein. As such, a cryptic peptide sequence may also be referred to as an insertion.

In some embodiments, the cryptic peptide sequence may be 1-50 amino acids in length. In some embodiments, the insertion may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length.

In some embodiments, the cryptic peptide sequence may be 1-50 amino acids in length.

In some embodiments, the cryptic peptide sequence may be 1-40 amino acids in length.

In some embodiments, the cryptic peptide sequence may be 1-30 amino acids in length.

In some embodiments, the cryptic peptide sequence may be 10-50 amino acids in length.

In some embodiments, the cryptic peptide sequence may be 10-40 amino acids in length.

In some embodiments, the cryptic peptide sequence may be 10-30 amino acids in length.

In some embodiments, the cryptic peptide sequence may be 10-20 amino acids in length.

In some embodiments, the cryptic peptide sequence may be 15-20 amino acids in length.

In some embodiments, the cryptic peptide sequence may be 18 amino acids in length.

In some embodiments, the cryptic peptide sequence may be 25-35 amino acids in length.

In some embodiments, the cryptic peptide sequence may be 29 amino acids in length.

In some embodiments, the cryptic peptide sequence may be located at any point within the splice variant of ICA1. In other words, the cryptic peptide sequence may be inserted at any position into the amino sequence of wild-type ICA1.

In some embodiments, the nucleic acid sequence encoding the cryptic peptide sequence may be inserted between exon 7 and exon 8 of the mRNA sequence encoding the splice variant of ICA1.

In some embodiments, the cryptic peptide sequence may be inserted at a position corresponding to position 302 of the amino acid sequence according to SEQ ID NO: 1 encoding wild-type ICA1.

In some embodiments, the cryptic peptide sequence may comprise or consist of the amino acid sequence: EGQQIGILISLEEENQRK (SEQ ID NO: 2). In some embodiments, the cryptic peptide sequence may comprise the amino acid sequence: EGQQIGILISLEEENQRK (SEQ ID NO: 2). In some embodiments, the cryptic peptide sequence may consist of the amino acid sequence:

(SEQ ID NO: 2)
EGQQIGILISLEEENQRK.

In some embodiments, the cryptic peptide sequence may comprise an amino acid sequence having at least 80%, 85%, 90%, 95%, 96%, 97%, 98%, 99%, or 100% sequence identity to the amino acid sequence: EGQQIGILISLEEENQRK (SEQ ID NO: 2). In some embodiments, the cryptic peptide sequence may consist of an amino acid sequence having at least 80%, 85%, 90%, 95%, 96%, 97%, 98%, 99%, or 100% sequence identity to the amino acid sequence:

(SEQ ID NO: 2)
EGQQIGILISLEEENQRK.

In some embodiments, the amino acid sequence EGQQIGILISLEEENQRK (SEQ ID NO: 2) may be encoded by a nucleic acid sequence having the sequence according to SEQ ID NO: 5.

SEQ ID NO: 5—nucleic acid sequence encoding EGQQIGILISLEEENQRK (SEQ ID NO: 2):

GAAGGCCAGCAAATTGGCATATTAATTTCATTAGAGGAAGAAAACCAG
CGCAAG.

In some embodiments, the cryptic peptide sequence may comprise or consist of the amino acid sequence: YLQNICLEMKANSDIKMLQKGKEGQQIGI (SEQ ID NO: 3). In some embodiments, the cryptic peptide sequence may comprise the amino acid sequence: YLQNICLEMKANSDIKMLQKGKEGQQIGI (SEQ ID NO: 3). In some embodiments, the cryptic peptide sequence may consist of the amino acid sequence:

(SEQ ID NO: 3)
YLQNICLEMKANSDIKMLQKGKEGQQIGI.

In some embodiments, the cryptic peptide sequence may comprise of an amino acid sequence having at least 80%, 85%, 90%, 95%, 96%, 97%, 98%, 99%, or 100% sequence identity to the amino acid sequence: YLQNICLEMKANSDIKMLQKGKEGQQIGI (SEQ ID NO: 3). In some embodiments, the cryptic peptide sequence may consist of an amino acid sequence having at least 80%, 85%, 90%, 95%, 96%, 97%, 98%, 99%, or 100% sequence identity to the amino acid sequence: YLQNICLEMKANSDIKMLQKGKEGQQIGI (SEQ ID NO: 3).

In some embodiments, the amino acid sequence YLQNICLEMKANSDIKMLQKGKEGQQIGI (SEQ ID NO: 3) may be encoded by a nucleic acid sequence having the sequence according to SEQ ID NO: 6.

SEQ ID NO: 6—nucleic acid sequence encoding YLQNICLEMKANSDIKMLQKGKEGQQIGI (SEQ ID NO: 3):

TATTTACAGAATATTTGCCTCGAGATGAAAGCAAATTCAGATATTAAGA
TGCTACAGAAAGGGAAAGAAGGCCAGCAAATTGGCATA.

It will be understood that, in some embodiments, for example due to protease digestion during mass spectrometry and sample preparation, the actual cryptic peptide sequence may be shorter in length than the cryptic peptide sequences described herein. In some embodiments, the actual cryptic peptide sequence may be shorter in length by 1, 2, 3, 4, 5, 6, 7, 8, 9, 10, 11, 12, 13, 14, 15 or more amino acids. In some embodiments, the actual cryptic peptide sequence may be shorter in length by 11 amino acids.

In some embodiments, for example, the actual cryptic peptide sequence may be “YLQNICLEMKANSDIKMLQKGKEGQQIGI” (SEQ ID NO: 3), but the peptide generated and detected may be “EGQQIGILISLEEENQRK” (SEQ ID NO: 2), where the 11 amino acids “LISLEEENQRK” belong to correctly spliced/canonical ICA1.

As such, in some embodiments, EGQQIGILISLEEENQRK (SEQ ID NO: 2) may be termed the proteotypic peptide, whereas the actual cryptic peptide sequence may be YLQNICLEMKANSDIKMLQKGKEGQQIGI (SEQ ID NO: 3).

In some embodiments, the splice variant of ICA1 may comprise or consists of an amino acid sequence according to SEQ ID NO: 4. In some embodiments, the splice variant of ICA1 may comprise an amino acid sequence according to SEQ ID NO: 4. In some embodiments, the splice variant of ICA1 may consist of an amino acid sequence according to SEQ ID NO: 4.

In some embodiments, the splice variant of ICA1 may comprise an amino acid sequence having at least 80%, 85%, 90%, 95%, 96%, 97%, 98%, 99%, or 100% sequence identity to the amino acid sequence according to SEQ ID NO: 4. In some embodiments, the splice variant of ICA1 may consist of an amino acid sequence having at least 80%, 85%, 90%, 95%, 96%, 97%, 98%, 99%, or 100% sequence identity to the amino acid sequence according to SEQ ID NO: 4.

SEQ ID NO: 4-amino acid sequence of the splice variant of ICA1:
MSGHKCSYPWDLQDRYAQDKSVVNKMQQKYWETKQAFIKATGKKEDEHVVASDADLDAKLE
LFHSIQRTCLDLSKAIVLYQKRICFLSQEENELGKFLRSQGFQDKTRAGKMMQATGKALCFSSQ
QRLALRNPLCRFHQEVETFRHRAISDTWLTVNRMEQCRTEYRGALLWMKDVSQELDPDLYKQ
MEKFRKVQTQVRLAKKNFDKLKMDVCQKVDLLGASRCNLLSHMLATYQTTLLHFWEKTSHTM
AAIHESFKGYQPYEFTTLKSLQDPMKKLVEKEEKKKINQQESTDAAVQEPSQYLQNICLEMKAN
SDIKMLQKGKEGQQIGILISLEEENQRKESSSFKTEDGKSILSALDKGSTHTACSGPIDELLDMK
SEEGACLGPVAGTPEPEGADKDDLLLLSEIFNASSLEEGEFSKEWAAVFGDGQVKEPVPTMAL
GEPDPKAQTGSGFLPSQLLDQNMKDLQASLQEPAKAASDLTAWFSLFADLDPLSNPDAVGKT
DKEHELLNA.

In some embodiments, the amino acid sequence of the splice variant of ICA1 (SEQ ID NO: 4) may be encoded by a nucleic acid sequence having the sequence according to SEQ ID NO: 7.

SEQ ID NO: 7-nucleic acid sequence encoding the splice variant of ICA1:
ATGTCAGGACACAAATGCAGTTATCCCTGGGACTTACAGGATCGATATGCTCAAGATAAGT
CAGTTGTAAATAAGATGCAACAGAAATATTGGGAGACGAAGCAGGCCTTTATTAAAGCCAC
AGGGAAGAAGGAAGATGAACATGTTGTTGCCTCTGACGCGGACCTGGATGCCAAGCTAGA
GCTGTTTCATTCAATTCAGAGAACCTGTCTGGACTTATCGAAAGCAATTGTACTCTATCAAA
AGAGGATATGTTTCTTGTCTCAAGAAGAAAACGAACTGGGAAAATTTCTTCGATCCCAAGGT
TTCCAAGATAAAACCAGAGCAGGAAAGATGATGCAAGCGACAGGAAAGGCCCTCTGCTTTT
CTTCCCAGCAAAGGTTGGCCTTACGAAATCCTTTGTGTCGATTTCACCAAGAAGTGGAGAC
TTTTCGGCATCGGGCCATCTCAGATACTTGGCTGACGGTGAACCGCATGGAACAGTGCAG
GACGGAATATAGAGGAGCACTATTATGGATGAAGGACGTGTCTCAGGAGCTTGATCCAGA
CCTCTACAAGCAAATGGAGAAGTTCAGGAAGGTACAAACACAAGTGCGCCTTGCAAAAAAA
AACTTTGACAAATTGAAGATGGATGTTTGTCAAAAAGTGGATCTTCTTGGAGCGAGCAGAT
GCAATCTCTTGTCTCACATGCTAGCAACATACCAGACCACTCTGCTTCATTTTTGGGAGAAA
ACTTCTCACACTATGGCAGCCATCCATGAGAGTTTCAAAGGTTATCAACCATATGAATTTAC
TACTTTAAAGAGCTTACAAGACCCTATGAAAAAATTAGTTGAGAAAGAAGAGAAGAAGAAAA
TCAACCAGCAGGAAAGTACAGATGCAGCCGTGCAGGAGCCGAGCCAATATTTACAGAATA
TTTGCCTCGAGATGAAAGCAAATTCAGATATTAAGATGCTACAGAAAGGGAAAGAAGGCCA
GCAAATTGGCATATTAATTTCATTAGAGGAAGAAAACCAGCGCAAGGAATCCTCTAGTTTTA
AGACTGAAGATGGAAAAAGTATTTTATCTGCCTTAGACAAAGGCTCTACACATACTGCATGC
TCAGGACCCATAGATGAACTATTAGACATGAAATCTGAGGAAGGTGCTTGCCTGGGACCAG
TGGCAGGGACCCCGGAACCTGAAGGTGCTGACAAAGATGACCTGCTGCTGTTGAGTGAGA
TCTTCAATGCTTCCTCCTTGGAAGAGGGCGAGTTCAGCAAAGAGTGGGCCGCTGTGTTTG
GAGACGGCCAAGTGAAGGAGCCAGTGCCCACTATGGCCCTGGGAGAGCCAGACCCCAAG
GCCCAGACAGGCTCAGGTTTCCTTCCTTCGCAGCTTTTAGACCAAAATATGAAAGACTTAC
AGGCCTCGCTACAAGAACCTGCTAAGGCTGCCTCAGACCTGACTGCCTGGTTCAGCCTCT
TCGCTGACCTCGACCCACTCTCAAATCCTGATGCTGTTGGGAAAACCGATAAAGAACACGA
ATTGCTCAATGCATGA.

### Biomarker

It will be understood that the term “biomarker” generally refers to an indicator of a biological state. The biomarker may be, for example, a molecule, a gene, a mRNA, a nucleic sequence, a peptide, a protein or an amino acid sequence. It will also be understood that a specific biomarker may be associated with a specific disease or pathological condition.

A biomarker may be used predictively as a means for identifying a subject who is at risk of developing a disease or pathological condition.

Alternatively, a biomarker may be used diagnostically as a means for identifying a subject who has a disease or pathological condition.

Alternatively, a biomarker may be used prognostically as a means for monitoring a disease or pathological condition in a subject, in particular a subject who has received and/or is the process of receiving a treatment for the disease or pathological condition. In this case, the presence or absence of the biomarker may indicate whether treatment of the disease or pathological condition is successful or not.

As used herein, the invention may comprise a biomarker. A biomarker according to the present invention is a splice variant of ICA1 according to the invention, wherein the ICA1 protein comprises a cryptic peptide sequence according to the invention.

It will be understood that the presence of the splice variant of ICA1 (i.e. the complete protein) and/or the cryptic peptide sequence (i.e. the isolated peptide) may be the biomarker.

In some embodiments, the biomarker is the splice variant of ICA1 according to the invention itself (i.e. the ICA1 protein comprising the cryptic peptide sequence).

In some embodiments, the biomarker is the cryptic peptide sequence (i.e. the isolated peptide) according to the invention.

In some embodiments, the biomarker may be in the form of a nucleic acid sequence, such as DNA, RNA, and/or mRNA encoding the splice variant of ICA1 or the cryptic peptide sequence.

In some embodiments, the biomarker may be an amino acid sequence, such as the amino acid sequence of the splice variant of ICA1 protein or the amino acid sequence of the cryptic peptide.

The biomarker may be termed a “signature”. For example, a biomarker in the form of a nucleic acid sequence may be a termed a “nucleic acid signature”, and a biomarker in the form of an amino acid sequence may be a termed an “amino acid signature”.

The expression of the biomarker relates to the amount of the biomarker that is present and/or detectable in a sample obtained from a subject. As such, the terms “expression”, “amount” or “level” can be considered as interchangeable in this regard.

Suitable methods for detecting and/or measuring the biomarker will be known in the art. Suitable methods for detecting and/or measuring the biomarker are disclosed herein.

### Diagnostic Methods

It will be understood that the methods according to the invention may typically be practiced outside of the human body or animal body. For example, the methods according to the invention may be performed using a sample obtained from a subject.

The presence of the cryptic peptide sequence according to the invention and/or a splice variant of ICA1, such as a splice variant of ICA1 comprising the cryptic peptide sequence according to the invention, can be used as a biomarker for a TDP-43 pathology. The presence of the cryptic peptide sequence according to the invention and/or the splice variant of ICA1 can additionally (or alternatively) be used as a biomarker to identify a cell, a tissue, or an organ of a subject having reduced TDP-43 function.

Accordingly, the present invention provides a method for identifying a TDP-43 pathology and/or reduced TDP-43 function in a subject, comprising the step of identifying a splice variant of ICA1 in a sample obtained from the subject. In some embodiments, identification of the splice variant of ICA1 involves identification of a cryptic peptide sequence. In some embodiments, the method is an ex vivo method. In some embodiments, the method is an in vitro method.

In some cases, individuals may have an undiagnosed TDP-43 pathology, for example they may have reduced or aberrant expression of TDP-43 in their neuronal cells but do not display symptoms of a TDP-43 pathology at that stage. As such, detecting the presence of a splice variant of ICA1 in a sample derived from such an individual may provide an indication that the individual is at risk of developing a TDP-43 pathology in the future.

Accordingly, the present invention provides a method of identifying a subject that is at risk of developing a TDP-43 pathology and/or reduced TDP-43 function, wherein the method comprises a step of identifying a splice variant of ICA1 in a sample obtained from the subject, wherein the presence of the splice variant of ICA1 in the sample indicates that the subject is likely to develop a TDP-43 pathology. In some embodiments, identification of the splice variant of ICA1 involves identification of a cryptic peptide sequence. In some embodiments, the method is an ex vivo method. In some embodiments, the method is an in vitro method.

In some cases, a patient may have been diagnosed with a TDP-43-related pathology (e.g. amyotrophic lateral sclerosis (ALS) or frontotemporal dementia), although the cause of the pathology (i.e. reduced TDP-43 function and/or reduced TDP-43 expression) may not be known. As such, detecting the presence of a splice variant of ICA1 in a sample derived from such a patient may be used to determine whether a patient has reduced TDP-43 function and/or reduced TDP-43 expression, and thus can consequently be used to determine whether a patient will be likely to benefit from a treatment that partially or completely restores TDP-43 function and/or reduced TDP-43 expression.

Accordingly, the present invention provides a method for determining whether a subject suffering from a TDP-43 pathology and/or reduced TDP-43 function is likely to respond to a treatment, wherein the method comprises a step of identifying a splice variant of ICA1 in a sample obtained from the subject, wherein the presence of the splice variant of ICA1 in the sample indicates that the subject is likely to respond to a treatment. In some embodiments, identification of the splice variant of ICA1 involves identification of a cryptic peptide sequence. In some embodiments, the method is an ex vivo method. In some embodiments, the method is an in vitro method.

During or following the administration of a treatment to treat a TDP-43 pathology and/or reduced TDP-43 function, the expression of the splice variant of ICA1 and/or the cryptic peptide sequence may become reduced or undetectable. Herein by “reduced” is meant that the amount or concentration of ICA1 and/or the cryptic peptide sequence detected in a patient sample following treatment is less than the amount or concentration of ICA1 and/or the cryptic peptide sequence detected in a patient sample prior to treatment.

Reduced expression or absence of a splice variant of ICA1 according to the invention and/or a cryptic peptide sequence according to the invention in a sample from a patient may be indicative that the treatment is (or has been) successful, for example due to rescuing of mis-spliced transcripts. This reduction may be compared relative to the expression or presence of the splice variant of ICA1 prior to administration of a treatment.

The appended examples demonstrate that cells not expressing TDP-43 express a splice variant of ICA1, but treatment of the cells with a (CA) n antisense oligonucleotide reduces expression of the splice variant of ICA1. These results indicate that the expression of a splice variant of ICA1 and/or a cryptic peptide sequence can be used for monitoring therapeutic success (e.g. of treatment that is ongoing or has been previously administered) during the treatment of a disease with a therapy that improves TDP-43 expression and/or TDP-43 function.

Accordingly, the present invention provides a method for monitoring the therapeutic success of a treatment of a TDP-43 pathology and/or reduced of TDP-43 function in a subject, wherein the method comprises a step of identifying a splice variant of ICA1 in a sample obtained from the subject; and further comprises a step of predicting the therapeutic success of the treatment of the TDP-43 pathology and/or reduced of TDP-43 function based on the detection of the splice variant of ICA1. In some embodiments, identification of the splice variant of ICA1 involves identification of a cryptic peptide sequence. In some embodiments, the method is an ex vivo method. In some embodiments, the method is an in vitro method.

In some embodiments, the absence of any detectable splice variant of ICA1 and/or cryptic peptide sequence may be indicative of therapeutic success (i.e. that a therapy is successful at treating a disease). In some embodiments, a reduction in detectable splice variant of ICA1 and/or cryptic peptide sequence may be indicative of therapeutic success (i.e. that a therapy is successful at treating a disease). The reduction may be a reduction by 5-100% in the expression of the splice variant of ICA1 and/or cryptic peptide sequence, compared with the expression of the splice variant of ICA1 and/or cryptic peptide sequence prior to the administration of the treatment. In some embodiments, the reduction may be by 10-100%, 20-100%, 30-100%, 40-100%, 50-100%, 60-100%, 70-100%, 80-100%, or 90-100%. In some embodiments, the reduction may be by 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the treatment may comprise a protein or a nucleic acid molecule capable of restoring the functional phenotype of one or more TDP-43 target RNA(s) in a cell which is TDP-43 depleted, or is expressing aberrant TDP-43 protein.

In some embodiments, the treatment may comprise an antisense oligonucleotide capable of restoring the functional phenotype of one or more TDP-43 target RNA(s) in a cell which is TDP-43 depleted, or is expressing aberrant TDP-43 protein. In some embodiments, the treatment may mimic the function of TDP-43 and rescue mis-spliced transcripts upon loss of TDP-43.

### USE

As described herein, loss of TDP-43 function and/or TDP-43 expression can result in the expression of a splice variant of ICA1 and/or cryptic peptide sequence. This splice variant of ICA1 and/or cryptic peptide sequence thus can be used as a biomarker that identifies a subject as having (or being at risk of developing) a TDP-43 pathology and/or reduced TDP-43 function.

Accordingly, the present invention provides a use of a splice variant of ICA1 as a biomarker for a TDP-43 pathology and/or reduced TDP-43 function. The present invention also provides a use of a cryptic peptide sequence as a biomarker for a TDP-43 pathology and/or reduced TDP-43 function.

In some embodiments, the presence of the biomarker in a sample obtained from a subject may be used to identify a subject as having a TDP-43 pathology and/or reduced TDP-43 function.

In some embodiments, the presence of the biomarker in a sample obtained from a subject may be used to identify a subject as being at risk of developing a TDP-43 pathology and/or reduced TDP-43 function.

In some embodiments, the presence or absence of the biomarker in a sample obtained from a subject may be used to determine whether a subject suffering from a TDP-43 pathology and/or reduced TDP-43 function is likely to respond to a treatment.

In some embodiments, the presence or absence of the biomarker in a sample obtained from a subject may be used to monitor the progression of a TDP-43 pathology and/or reduced TDP-43 function in a subject.

In some embodiments, the presence or absence of the biomarker in a sample obtained from a subject may be used to monitor the treatment of a TDP-43 pathology and/or reduced TDP-43 function in a subject, who has been received and/or is the process of receiving a treatment for the TDP-43 pathology and/or reduced TDP-43 function.

### Identifying the Splice Variant of ICA1

It will be understood that a TDP-43 pathology and/or reduced TDP-43 function may be identified: (i) by detecting the splice variant of ICA1 according to the invention; (ii) by detecting a cryptic peptide sequence that is comprised within the splice variant of ICA1 according to the invention; and/or (iii) by detecting the cryptic peptide sequence according to the invention itself, which has been isolated from the splice variant of ICA1 according to the invention.

In some embodiments, the full splice variant of ICA1 protein (i.e. comprising the cryptic peptide sequence) may be detected. In some embodiments, the cryptic peptide sequence (i.e. isolated from the full splice variant of ICA1 protein) may be detected.

In some embodiments, the expression of the cryptic peptide sequence according to the invention and/or the splice variant of ICA1 according to the invention may be measured or detected at the level of mRNA expression (i.e. at the transcript level), by measuring or detecting one or more nucleic acid sequence(s) encoding the cryptic peptide sequence according to the invention and/or the splice variant of ICA1 according to the invention.

Expression of mRNA encoding the cryptic peptide sequence according to the invention and/or the splice variant of ICA1 according to the invention may be measured or detected using any method or technique known in the art that is suitable for measuring mRNA or transcript expression. Suitable techniques include quantitative PCR (including quantitative PCR) and RNA sequencing.

In some embodiments, the expression of the cryptic peptide sequence according to the invention and/or the splice variant of ICA1 according to the invention may be measured or detected at the level of protein expression.

Protein expression of the cryptic peptide sequence according to the invention and/or the splice variant of ICA1 according to the invention may be measured or detected using any method or technique known in the art that is suitable for measuring protein expression. Suitable techniques include proteomic analysis and antibody based assays. For example, protein expression of the cryptic peptide sequence according to the invention and/or the splice variant of ICA1 according to the invention may be measured or detected using proteomics, mass spectrometry (MS), chromatography, enzyme-linked immunoabsorbent assay (ELISA), flow cytometry, Western blotting or an ultra-sensitive immunoassay (see e.g. https://www.quanterix.com/simoa-technology/). Mass spectrometry may encompass any form of mass spectrometry, including (but not limited to) hyper reaction monitoring mass spectrometry (HRM-MS).

It will be understood that global proteomic profiling using mass spectrometry is suitable for use with the present invention to identify the splice variant of ICA1 according to the invention and/or the cryptic peptide sequence according to the invention. For example, digestion of a sample with Lys-C protease followed by hyper reaction monitoring mass spectrometry (HRM-MS) protein profiling may be used to identify the splice variant of ICA1 according to the invention and/or the cryptic peptide sequence according to the invention.

In some embodiments, the step of identifying the splice variant of ICA1 and/or the cryptic peptide sequence may comprise proteomic analysis. In some embodiments, the proteomic analysis is performed using a sample obtained from a subject.

In some embodiments, the step of identifying the splice variant of ICA1 and/or the cryptic peptide sequence comprises measuring peptides in a sample using liquid chromatography (LC). In some embodiments, the step of identifying the splice variant of ICA1 and/or the cryptic peptide sequence comprises measuring peptides in a sample using mass spectrometry (MS). In some embodiments, the mass spectrometry is hyper reaction monitoring mass spectrometry (HRM-MS).

In some embodiments, the step of identifying the splice variant of ICA1 and/or the cryptic peptide sequence may comprise an antibody-based assay. In some embodiment, the antibody-based assay may be ELISA, flow cytometry, or Western blotting.

### Sample

As described herein, the cryptic peptide sequence according to the invention and/or the splice variant of ICA1 according to the invention may be detected in a sample obtained from a subject.

In some embodiments, the sample is obtained from a bodily fluid or tissue. In some embodiments, the sample is selected from: blood, serum, plasma, urine, saliva, brain tissue and cerebrospinal fluid (CSF).

In some embodiments, the sample is blood.

In some embodiments, the sample is brain tissue.

In some embodiments, the sample is cerebrospinal fluid (CSF).

Methods and techniques for obtaining these samples from a subject are known in the art.

Any method or technique that is suitable for obtaining these samples from a subject is encompassed by the present invention.

### Subject

In some embodiments, the subject of the methods and uses according to the present invention may be a mammal.

In some embodiments, the subject may be a human.

In other embodiments, the subject may alternatively be a non-human mammal, e.g. a dog, a cat, a horse, a cow, or a pig.

In some embodiments, the subject is a patient. In some embodiments, the subject is a human patient.

The subject may be a patient in the process of receiving a treatment (i.e. on-going therapy), such as a treatment to treat a TDP-43 pathology and/or reduced TDP-43 function. The subject may be a patient that has previously received a treatment, such as a treatment to treat a TDP-43 pathology and/or reduced TDP-43 function.

As used herein, it will be understood that the terms “patient” and “subject” are considered to be interchangeable.

### Disease

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. In some embodiments, the treatment may not be prophylactic, for example the treatment is treatment of an existing disease condition which has been diagnosed in the patient.

It will be understood that the terms “pathology” and “disease” are interchangeable.

It will be understood that the terms “treatment” and “therapy” are interchangeable.

A TDP-43 pathology is a disease which is associated with reduced or aberrant expression of TDP-43, often associated by an increase in cytoplasmic TDP-43, particularly hyper-phosphorylated and ubiquitinated TDP-43.

In some embodiments, the TDP-43 pathology and/or reduced TDP-43 function may be caused by a neuronal cell or neuronal cells that express a disease-associated variant of TDP-43, and/or express dysfunctional TDP-43 and/or are depleted in normal TDP-43 protein.

TDP-43 depletion is indicated in a range of diseases, referred to as TDP-43 pathologies.

In some embodiments, the TDP-43 pathology may be a neurological disorder. In some embodiments, the reduced TDP-43 function may be associated with a neurological disorder.

In some embodiments, the neurological disorder may be selected from: amyotrophic lateral sclerosis (ALS), frontotemporal dementia, frontotemporal lobar degeneration (FTLD), progressive supranuclear palsy (PSP), primary lateral sclerosis, progressive muscular atrophy, alzheimer's disease, parkinson's disease, autism, hippocampal sclerosis dementia, down syndrome, huntington's disease, polyglutamine diseases, such as spinocerebellar ataxia 3, myopathies and chronic traumatic encephalopathy.

In some embodiments, the neurological disorder may be selected from: amyotrophic lateral sclerosis (ALS), frontotemporal dementia, and frontotemporal lobar degeneration (FTLD). In some embodiments, the neurological disorder may be amyotrophic lateral sclerosis (ALS). In some embodiments, the neurological disorder may be frontotemporal dementia. In some embodiments, the neurological disorder may be frontotemporal lobar degeneration (FTLD).

### Antibody

The present invention provides an antibody that binds to a splice variant of ICA1 according to the invention.

In some embodiments, the antibody may bind to the cryptic peptide sequence according to the invention comprised within the amino acid sequence of the splice variant of ICA1 according to the invention.

In some embodiments, the antibody may bind to the cryptic peptide sequence according to the invention which is isolated from the full sequence of the splice variant of ICA1 according to the invention.

In some embodiments, the antibody may bind exclusively to the cryptic peptide sequence according to the invention.

In some embodiments, the epitope that the antibody may bind to may be the cryptic peptide sequence comprising or consisting of the amino acid sequence: EGQQIGILISLEEENQRK (SEQ ID NO: 2), or an amino acid sequence having at least 80%, 85%, 90%, 95%, 96%, 97%, 98%, 99%, or 100% sequence identity thereto.

In some embodiments, the epitope that the antibody may bind to may be the cryptic peptide sequence comprising or consisting of the amino acid sequence: YLQNICLEMKANSDIKMLQKGKEGQQIGI (SEQ ID NO: 3), or an amino acid sequence having at least 80%, 85%, 90%, 95%, 96%, 97%, 98%, 99%, or 100% sequence identity thereto.

In some embodiments, the antibody may bind to part of (e.g. the epitope may over lap with) the cryptic peptide sequence according to the invention. In some embodiments, the epitope that the antibody may bind to may comprise part of the cryptic peptide sequence comprising or consisting of the amino acid sequence: YLQNICLEMKANSDIKMLQKGKEGQQIGI (SEQ ID NO: 3), or an amino acid sequence having at least 80%, 85%, 90%, 95%, 96%, 97%, 98%, 99%, or 100% sequence identity thereto.

### Kit

The present invention provides a kit comprising at least one reagent for detecting a splice variant of ICA1 according to the invention, wherein the at least one reagent is an antibody according to the invention.

In some embodiments, the kit may comprise instructions for using the kit.

In some embodiments, the kit may comprise additional reagents.

### General Terms and Definitions

The term “polypeptide” is used in the conventional sense to mean a series of amino acids, typically L-amino acids, connected one to the other, typically by peptide bonds between the α-amino and carboxyl groups of adjacent amino acids. The term “polypeptide” is used interchangeably with the terms “amino acid sequence”, “peptide” and/or “protein”. The term “residues” is used to refer to amino acids in an amino acid sequence.

The term “variant” refers to a polypeptide that has an equivalent function to the amino acid sequences described herein, but which includes one or more amino acid substitutions, insertions or deletions.

As used herein, “variant” is synonymous with “mutant” and refers to a polynucleotide or amino acid sequence which differs in comparison to the corresponding wild-type sequence. The term “wild-type” is used to mean a gene or protein having a polynucleotide or amino acid sequence respectively, which is identical with the native gene or protein respectively.

A nucleic acid sequence may be an RNA or DNA sequence or a variant thereof. The term “polynucleotide” includes an RNA or DNA sequence. It may be single or double stranded. It may, for example, be genomic, recombinant, mRNA or cDNA.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### Identity

The terms “identity” and “% sequence identity” as used herein, may refer to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Similarly, the terms “identity” and “% sequence identity” as used herein, may refer to the proportion of amino acids (expressed in percent) of an amino acid sequence in a peptide or protein, which across the amino acid sequence, are identical to a reference sequence. The percentage of identity is thus calculated by counting the number of aligned amino acids that are identical (a Match) between two sequences (in the amino acids sequence of the peptide or protein of the invention and in the reference sequence), dividing that number by the total number of amino acids in the amino acid sequence and multiplying by 100. Therefore, Percentage of Identity=(Matches×100)/Length of aligned region. Insertions and deletions are not allowed in the calculation the percentage of identity of an amino acid sequence.

SUMMARY OF SEQUENCES

| SEQ ID NO | Description |
|---|---|
| 1 | wild-type ICA1 protein |
| 2 | EGQQIGILISLEEENQRK cryptic peptide sequence |
| 3 | YLQNICLEMKANSDIKMLQKGKEGQQIGI cryptic peptide |
| | sequence |
| 4 | splice variant of ICA1 protein |
| 5 | Nucleic acid sequence encoding EGQQIGILISLEEENQRK |
| 6 | Nucleic acid sequence encoding |
| | YLQNICLEMKANSDIKMLQKGKEGQQIGI |
| 7 | Nucleic acid sequence encoding splice variant of ICA1 |

### EXAMPLES

### Example 1

### Methods

### HRM Mass Spectrometry

Commercially available hiPSC-derived glutamatergic cortical neurons, iCell GlutaNeurons (FUJIFILM Cellular Dynamics, Cat. R1034), were co-cultured with iCell Astrocytes (FUJIFILM Cellular Dynamics, Cat. R1092) following the manufacturer's guidelines. The ratio of astrocytes to neurons was 1:4. Cells were treated with corresponding antisense LNA gapmer or (CA) n antisense oligonucleotide (ASO) on day 2 and day 5, respectively. Media change was performed every 3-4 days with either LNA gapmer or (CA) n ASO. For TDP-43 KD cells, only LNA gapmer was added every second media change. Cells were harvested and pelleted on day 30. Cell pellets were frozen and kept at −80° C. Each sample contains approximately 2 million cells.

Global proteomic profiling using mass spectrometry (MS) was done at Biognosys AG (Schlieren, Switzerland). In brief, frozen cell pellets were lysed and further digested with Lys-C protease. Hyper reaction monitoring (HRM)-MS protein profiling was performed. The data was analyzed using Spectronaut 18 Pulsar software.

### ASOs Used in the Study

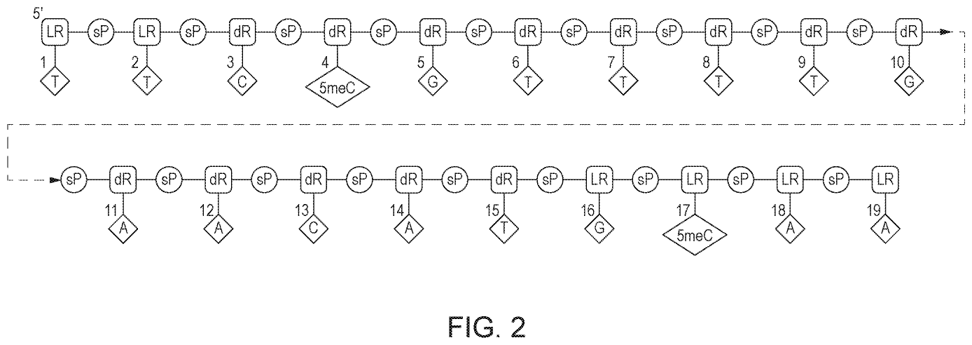

1. LNA gapmer (used for knocking down TDP-43): 5′-TTCCGTTTTGAACATGCAA-3′ The structure of the LNA gapmer is shown in FIG. 2.
2. (CA) n ASO (used for mimicking the function of TDP-43 and rescuing misspliced transcripts upon loss of TDP-43):

5′-[CholTEG]-CACACACACACACACACACACACAC-3′

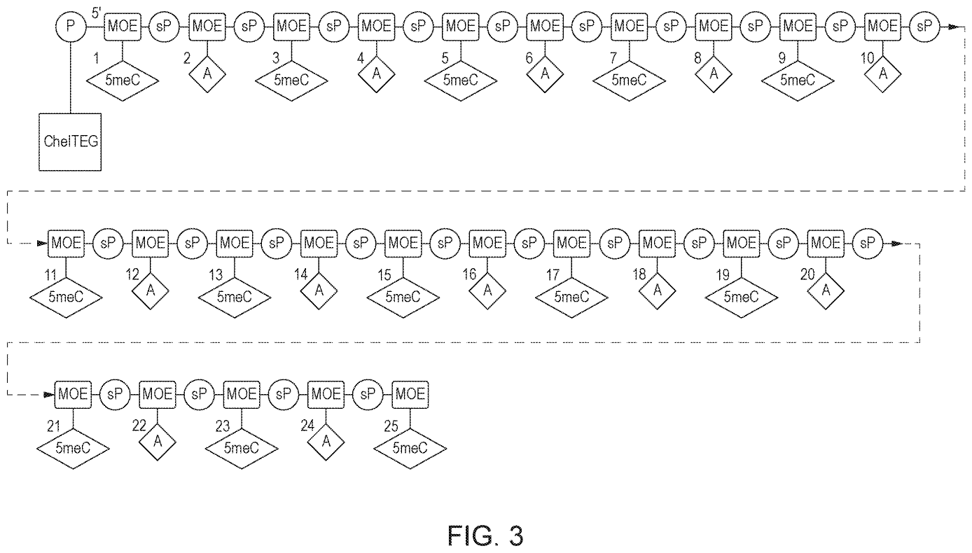

The structure of the (CA) n ASO is shown in FIG. 3.

### Results

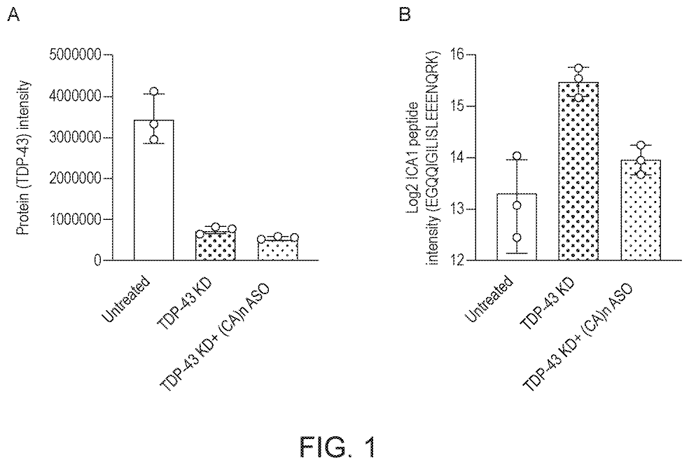

Knockdown of TDP-43 in co-cultures of hiPSC-derived neurons and astrocytes was performed using the LNA gapmer. The TDP-43 level was reduced by approximately 80% (FIG. 1A).

Upon loss of TDP-43, mass spectrometry revealed a remarkable increase in the level of the novel proteotypic peptide EGQQIGILISLEEENQRK. The peptide intensity was greatly reduced when treated with (CA) n ASO (FIG. 1B).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for identifying a TDP-43 pathology and/or reduced TDP-43 function in a subject, comprising the step of identifying a splice variant of ICA1 in a sample obtained from the subject.

2. A method of identifying a subject that is at risk of developing a TDP-43 pathology and/or reduced TDP-43 function, wherein the method comprises a step of identifying a splice variant of ICA1 in a sample obtained from the subject, wherein the presence of the splice variant of ICA1 in the sample indicates that the subject is likely to develop a TDP-43 pathology.

3. A method for determining whether a subject suffering from a TDP-43 pathology and/or reduced TDP-43 function is likely to respond to a treatment, wherein the method comprises a step of identifying a splice variant of ICA1 in a sample obtained from the subject, wherein the presence of the splice variant of ICA1 in the sample indicates that the subject is likely to respond to a treatment.

4. A method for monitoring the therapeutic success of a treatment of a TDP-43 pathology and/or reduced TDP-43 function in a subject, wherein the method comprises a step of identifying a splice variant of ICA1 in a sample obtained from the subject; and further comprises a step of predicting the therapeutic success of the treatment of the TDP-43 pathology and/or reduced TDP-43 function based on the detection of the splice variant of ICA1.

5. The method according to claim 3 or claim 4, wherein the treatment comprises an antisense oligonucleotide capable of restoring the functional phenotype of one or more TDP-43 target RNA(s) in a cell which is TDP-43 depleted, or is expressing aberrant TDP-43 protein.

6. Use of a splice variant of ICA1 as a biomarker for a TDP-43 pathology and/or reduced TDP-43 function.

7. The method according to any one of claims 1-5, or the use according to claim 6, wherein the splice variant of ICA1 comprises a cryptic peptide sequence.

8. The method or use according to claim 7, wherein the splice variant of ICA1 comprises a cryptic peptide sequence of 10-50 amino acids.

9. The method or use according to claim 8, wherein the splice variant of ICA1 comprises a cryptic peptide sequence of 25-35 amino acids.

10. The method or use according to claim 8 or claim 9, wherein the splice variant of ICA1 comprises a cryptic peptide sequence of 29 amino acids.

11. The method or use according to any one of claims 7-10, wherein the cryptic peptide sequence is inserted at a position corresponding to position 302 of the amino acid sequence according to SEQ ID NO: 1 encoding wild-type ICA1.

12. The method or use according to any one of claims 7-11, wherein the cryptic peptide sequence comprises or consists of the amino acid sequence YLQNICLEMKANSDIKMLQKGKEGQQIGI (SEQ ID NO: 3), or a variant thereof having at least 80% sequence identity thereto.

13. The method or use according to claim 12, wherein the cryptic peptide sequence comprises or consists of an amino acid sequence having at least 85%, 90%, 95%, 96%, 97%, 98%, 99%, or 100% sequence identity to the amino acid sequence YLQNICLEMKANSDIKMLQKGKEGQQIGI (SEQ ID NO: 3).

14. The method or use according to claim 12 or claim 13, wherein the cryptic peptide sequence comprises or consists of the amino acid sequence YLQNICLEMKANSDIKMLQKGKEGQQIGI SEQ ID NO: 3.

15. The method according to any one of claim 1-5 or 7-14, or the use according to any one of claims 6-14, wherein the splice variant of ICA1 comprises or consists of an amino acid sequence according to SEQ ID NO: 4 or a variant with at least 80% sequence identity thereto.

16. The method or use according to claim 15, wherein the splice variant of ICA1 comprises or consists of an amino acid having at least 85%, 90%, 95%, 96%, 97%, 98%, 99%, or 100% sequence identity to SEQ ID NO: 4.

17. The method or use according to claim 15 or claim 16, wherein the splice variant of ICA1 comprises or consists of the amino acid sequence according to SEQ ID NO: 4.

18. The method according to any one of claim 1-5 or 7-17, wherein the method is an ex vivo method.

19. The method according to any one of claim 1-5 or 7-18, wherein the step of identifying the splice variant of ICA1 comprises proteomic analysis or an antibody based assay.

20. The method according to any one of claim 1-5 or 7-19, wherein the step of identifying the splice variant of ICA1 comprises measuring peptides in the sample using liquid chromatography (LC) and/or mass spectrometry (MS).

21. The method according to any one of claim 1-5 or 7-20, wherein the sample is obtained from a bodily fluid or tissue, selected from: blood, serum, plasma, urine, saliva, brain tissue and cerebrospinal fluid (CSF).

22. The method according to any one of claim 1-5 or 7-21, wherein the subject is human, preferably wherein the subject is a human patient.

23. The method according to any one of claim 1-5 or 7-22, or the use according to any one of claims 6-17, wherein the TDP-43 pathology is a neurological disorder.

24. The method or the use according to claim 23, wherein the TDP-43 pathology is a neurological disorder selected from: amyotrophic lateral sclerosis (ALS), frontotemporal dementia, frontotemporal lobar degeneration (FTLD), progressive supranuclear palsy (PSP), primary lateral sclerosis, progressive muscular atrophy, alzheimer's disease, parkinson's disease, autism, hippocampal sclerosis dementia, down syndrome, huntington's disease, polyglutamine diseases, such as spinocerebellar ataxia 3, myopathies and chronic traumatic encephalopathy.

25. The method or the use according to claim 23 or claim 24, wherein the TDP-43 pathology is a neurological disorder selected from: amyotrophic lateral sclerosis (ALS), frontotemporal dementia, and frontotemporal lobar degeneration (FTLD).

26. An antibody that binds to a splice variant of ICA1.

27. The antibody according to claim 26, wherein the splice variant of ICA1 comprises an epitope comprising or consisting of the amino acid sequence YLQNICLEMKANSDIKMLQKGKEGQQIGI (SEQ ID NO: 3), or a variant thereof having at least 80% sequence identity to thereto.

28. The antibody according to claim 26 or claim 27, wherein the splice variant of ICA1 comprises an epitope comprising or consisting of an amino acid sequence having at least 85%, 90%, 95%, 96%, 97%, 98%, 99%, or 100% sequence identity to the amino acid sequence YLQNICLEMKANSDIKMLQKGKEGQQIGI (SEQ ID NO: 3).

29. The antibody according to any one of claims 26-28, wherein the splice variant of ICA1 comprises or consists of an amino acid sequence according to SEQ ID NO: 4 or a variant with at least 80% sequence identity to SEQ ID NO: 4.

30. The antibody according to any one of claims 26-29, wherein the splice variant of ICA1 comprises or consists of an amino acid having at least 85%, 90%, 95%, 96%, 97%, 98%, 99%, or 100% sequence identity to SEQ ID NO: 4.

31. A kit comprising at least one reagent for detecting a splice variant of ICA1, wherein the at least one reagent is an antibody according to any one of claims 26-30.
